(19) United States
(12) Patent Application Publication
(10) Pub. No.: US 2011/0109458 A1
(43) Pub. Date: May 12, 2011

(54) Endotracheal Tube Cuff Pressure Measuring Device

(76) Inventor: Nolan D. Shipman, Bryan, TX (US)

(21) Appl. No.: 12/613,981

(22) Filed: Nov. 6, 2009

(51) Int. Cl. G08B 23/00; A61M 16/04

(52) U.S. Cl. 340/573.1; 128/207.14

(57) ABSTRACT

The invention relates to methods and devices related to improvements in the use of a medical breathing tube. In some embodiments, the invention reduces injuries, diseases and death associated with the use of said breathing tube. In preferred embodiments, said breathing tube reduces the risk of inadequate inflation in the lungs.

### FIELD OF INVENTION

The invention relates to methods and devices related to improvements in the use of a medical breathing tube. In some embodiments, the invention reduces injuries and diseases associated with the use of said breathing tube. In preferred embodiments, the present invention provides methods and devices to ensure proper pressure of an endotracheal tube cuff.

### BACKGROUND OF THE INVENTION

An endotracheal tube, also known as a breathing tube, is commonly used to maintain an unobstructed pathway to a patient's lungs, typically in the context of mechanical ventilation. The tube may be further supplemented with an endotracheal tube cuff, an apparatus that aids in the operation of an endotracheal tube, e.g. in the prevention of leaks in the ventilating circuit.

Maintaining endotracheal tube cuff pressures are important. Excessively high pressure in the cuff causes tracheal wall injury, while low pressure allows fluids to flow down the trachea into the lung and may result in diseases and disorders including but not limited to ventilator-associated pneumonia. The aforementioned disorders are difficult to treat and may result in death. Thus, there is a need to measure and correctively regulate tracheal tube cuff pressure.

### SUMMARY OF THE INVENTION

The invention relates to methods and devices related to improvements in the use of a medical breathing tube. In some embodiments, the invention reduces injuries and diseases associated with the use of said breathing tube. In preferred embodiments, the present invention provides methods and devices to ensure proper pressure of an endotracheal tube cuff, e.g. by continuously monitoring cuff pressure and alerting medical staff when the pressure is not optimum (e.g. not in the desired range of between 20-30 cm H₂O, and more preferably between 20-25 cm H₂O). In one embodiment, medical staff are alerted by an alarm (e.g. sound or, more preferably, a visual alarm, e.g. a red light and/or blinking light). Thereafter, the medical staff can manually adjust the cuff pressure (up or down) using an inflating/deflating means, in order to return the pressure to optimum levels. In one embodiment, the cuff pressure is indicated on a display (e.g. a computer screen) located away from (e.g. outside the patient's room, e.g. at the nurse's station) the endotracheal tube. In one embodiment, an alarm indicating undesired cuff pressure is located away from (e.g. outside the patient's room, e.g. at the nurse's station) the endotracheal tube. Continuously monitoring cuff pressure is preferred over “spot checking” at intervals because the latter results in significant delays before improper pressures are detected.

In one embodiment, the present invention contemplates a device comprising: an endotracheal tube cuff in fluid communication with a pressure sensor and a cuff inflating/deflating means, said pressure sensor in electronic communication with a comparator, said comparator in electronic communication with an alarm. In operation, said pressure sensor generates a voltage, and said voltage is transmitted to said comparator. In a preferred embodiment, an amplifier is positioned between said pressure sensor and said comparator, in order that said voltage of said pressure sensor is amplified, and said amplified voltage is transmitted to said comparator as input. In a preferred embodiment, said comparator is dual comparator. In another preferred embodiment, said comparator comprises Schmitt triggers.

It is not intended that the present invention be limited by the nature of the inflating/deflating means. In one embodiment, a communicating tube extends along all or part of the endotracheal tube, in fluid communication the cuff for inflating thereof. In one embodiment, the communicating tube terminates in a an inflation valve coupling for connecting the communicating tube and in turn the cuff to the other elements of the device as described herein. In one embodiment, the communicating tube terminates in a an inflation valve coupling which in turn connects via connecting the delivery tube and terminates in a coupling which in turn connects in fluidic communication to a three port stopcock/three-way manifold. In one embodiment, the three port stopcock connects in fluidic communication through a coupling and in turn through a delivery tube and terminates in an inflation valve coupling. In one embodiment, a syringe connects in fluidic communication to the inflation valve coupling to the delivery tube. In one embodiment, the three port stopcock connects in fluidic communication through coupling and in turn through a delivery tube and terminates in a Pressure Sensor.

In some embodiments, the invention relates to a device comprising: an endotracheal tube cuff connected to a tube comprising a polymer appropriate for use in medical applications (e.g. polyimide, ethylene vinyl acetate, etc.) subsequently connected to a manifold which is connected to (e.g. slidably engaging) both a syringe and a pressure sensor (e.g. the tubing may slide over a port or other opening in a housing containing the pressure sensor). The pressure sensor is subsequently connected to an amplifier appropriate for use in medical equipment. The amplifier is connected to both a comparator circuit and a data acquisition instrument. The comparator is subsequently connected to a light emitting diode and the data acquisition instrument is connected to computer operably linked to said data acquisition instrument. In further embodiments, said tube is an endotracheal tube. In still further embodiments, said light emitting diode emits red light and green light. In additional embodiments, said data acquisition instrument is a National Instruments USB 6008. In some embodiments, said computer operably linked to said data acquisition instrument is a National Instruments LabView computer.

The invention contemplates the above-described embodiments of the device operating as a “system,” as well as the device coupled to a ventilation circuit, thereby creating a coupled system for controlling cuff pressure an inflatable cuff of an endotracheal tube of an intubated patient.

In some embodiments, the invention relates to a method for treating a disease or disorder comprising: providing a subject at risk for or exhibiting symptoms associated with said disease or disorder, an embodiment of the device as described herein (e.g. an endotracheal tube cuff connected to a tube comprising a polymer appropriate for use in medical applications subsequently connected to a manifold which is connected to both a syringe and a pressure sensor) and administering said device under conditions such that the symptoms associated with said disease are reduced. In further embodiments, said disease or disorder is selected from the group consisting of pulmonary disease, pneumonia, pneumothorax, excess lung pressure, inadequate lung pressure, tooth damage, soft tissue damage, vocal chord damage, acute respiratory distress syndrome, tracheal rupture, tracheo-carotid artery erosion and tracheal innominate artery fistulas.

In some embodiments, the invention relates to a system comprising: an endotracheal tube cuff in fluid communication with a pressure sensor and a cuff inflating/deflating means, said pressure sensor capable of generating voltage and in electronic communication with a comparator, said comparator in electronic communication with an alarm.

In some embodiments, the invention further relates to a system wherein an amplifier is positioned between said pressure sensor and said comparator, in order that said voltage of said pressure sensor is amplified, and in order that said amplified voltage is transmitted to said comparator as input. In some embodiments, the invention further relates to a system wherein said comparator is dual comparator. In some embodiments, the invention further relates to a system of wherein said comparator comprises Schmitt triggers. In some embodiments, the invention further relates to a system wherein said endotracheal tube cuff is attached to an endotracheal tube positioned in a patient. In some embodiments, the invention further relates to a system wherein said alarm is a visual alarm. In some embodiments, the invention further relates to a system wherein said alarm is remote from said tube cuff. In some embodiments, the invention further relates to a system wherein said alarm is visible from a nurse's station.

In some embodiments, the invention relates to a method comprising: a) continuously monitoring cuff pressure of endotracheal tube cuff with a pressure sensor, said tube cuff attached to a endotracheal tube, said tube positioned in a patient, said pressure sensor capable of generating voltage and in electronic communication with a comparator, said comparator in electronic communication with an alarm; and b) alerting medical staff when the pressure is not optimum (e.g. too high or too low) with said alarm. In some embodiments, the invention further relates to a method wherein said alarm is a visual alarm. In some embodiments, the invention further relates to a method wherein said alarm is remote from said tube cuff. In some embodiments, the invention further relates to a method wherein said alarm is visible from a nurse's station and said medical staff are alerted at said nurse's station. In some embodiments, the invention further relates to a method wherein said pressure sensor is also in fluid communication with a cuff inflating/deflating means. In some embodiments, the invention further relates to a method wherein said medical staff adjust said pressure with said inflating/deflating means after being alerted in step b). In some embodiments, the invention further relates to a method wherein said adjusting comprises pushing or pulling on a syringe, said syringe in fluid communication with said tube cuff. In some embodiments, the invention further relates to a method wherein said monitoring of step a) comprises generating a voltage with said pressure sensor, and transmitting said voltage to said comparator.

In some embodiments, the invention further relates to a method for treating a disease or disorder comprising: a) providing: i) a subject intubated with an endotracheal tube at risk for or exhibiting symptoms associated with said disease or disorder (e.g. at risk because of the tube or tube cuff, including because of an improper pressure of the tube cuff), and ii) an embodiment of the device described herein (e.g. an endotracheal tube cuff in fluid communication with a pressure sensor and a cuff inflating/deflating means); b) administering said device under conditions such that the symptoms associated with said disease are reduced. In some embodiments, the said disease or disorder is a result of over-inflation of the endotracheal tube cuff. In some embodiments, the said disease or disorder is a result of under-inflation of the endotracheal tube cuff. In some embodiments, over-inflation of the endotracheal tube cuff is when endotracheal tube cuff is above 30 cm H₂O. In some embodiments, under-inflation of the endotracheal tube cuff is when endotracheal tube cuff is below 20 cm H₂O.

In some embodiments, the invention further relates to a method wherein said disease or disorder is selected from the group consisting of pulmonary disease, pneumonia, pneumothorax, excess lung pressure, inadequate lung pressure, tooth damage, soft tissue damage, vocal chord damage, acute respiratory distress syndrome, tracheal rupture, tracheo-carotid artery erosion and tracheal innominate artery fistulas.

In some embodiments, the device continuously measures the pressure of multiple cuffs (or multiple devices are used together with multiple cuffs, each device monitoring one cuff), including double-cuff tubes as described in U.S. Pat. No. 5,033,466, hereby incorporated by reference.

### DEFINITIONS

To facilitate the understanding of this invention, a number of terms are defined below. Terms defined herein have meanings as commonly understood by a person of ordinary skill in the areas relevant to the present invention.

One element is in “fluid communication” or “fluidic communication” with another element (and thereby “connected” to another element) when it is attached through a channel, tube or other conduit that permits the passage of gas, vapor and the like. Indeed, the tubing associated with commercially available ventilators creates a “circuit” for gas flow by maintaining fluidic communication between the elements of the circuit. Ports in the circuit allow for the circuit to be completed with tubing. “Tubing” can be made of a variety of materials, including put not limited to various plastics, metals and composites. Tubing can be rigid or flexible. Tubing can be “attached” in a detachable mode or a fixed mode. Tubing is typically attached by sliding into or over (both of which are examples of “slidably engaging”) other tubing or connectors (also called “couplings”).

In some embodiments, certain elements are in electronic communication with other elements (and thereby “communicate electronically”). “Electronic communication” can be implemented in a hard-wired electrical connection, e.g., a shielded cable, or an optical connection, e.g., an optical fiber, a wireless communication, e.g., infrared or radiowaves, a combination thereof, and the like.

In general, a comparator is a device which compares two voltages or currents and switches its output to indicate which is larger. A Schmitt trigger is a comparator circuit which “triggers” a change in output when the input changes sufficiently to warrant a change. When the input is higher than a certain chosen threshold, the output is high; when the input is below another (lower) chosen threshold, the output is low; when the input is between the two, the output retains its value. The “trigger” is so named because the output retains its value until the input changes sufficiently to trigger a change.

As used herein, “endotracheal tube” or “breathing tube” refers to a device used to aid the airway management and mechanical ventilation of a subject under anesthesia, intensive care or emergency medical care, including but in no way limited to a subject who are undergoing or who have recently undergone surgery including but not limited to thoracic surgery, a subject who has experienced a physical trauma including but not limited to thoracic and cardiothoracic trauma, a subject under the influence of at least one local or general anesthesia, or a subject experiencing loss of consciousness including but not limited to a subject in a medically induced or non-medically induced coma. The act of inserting an endotracheal tube or breathing tube is referred to as “intubation.”

An “endothracheal tube cuff” is an apparatus operably linked to an endotracheal tube capable of manipulating the pressure and volume of gas transferred into a patient using an endotracheal tube. Such cuffs are described generally in U.S. Pat. No. 5,067,497, hereby incorporated by reference.

“Pneumothorax,” also known as “collapsed lung,” is a condition caused by the accumulation of air or gas in the pleural cavity. While not limiting the present invention to the conditions under which a subject acquires pneumothorax, the condition may result from a disease or from physical injury.

“Subject” refers to any mammal, preferably a human patient.

As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

### BRIEF DESCRIPTION OF THE DRAWINGS

FIG. 1 is a schematic showing one embodiment of the device of the present invention in the context of a ventilation circuit, thereby creating a cuff pressure control coupled system.

FIG. 2 is a photograph of one embodiment of the device wherein the indicator light (alarm) indicates improper cuff pressure.

FIG. 3 is a photograph of the embodiment shown in FIG. 2, except that the indicator light (alarm) indicates proper cuff pressure.

FIG. 4 shows a block diagram detailing the components comprising one embodiment of the device of the present invention.

FIG. 5 shows the design flow chart of one embodiment of the device of the present invention.

FIG. 6 shows a design flow chart for potential failures and appropriate corrective actions for said failures related to the use of an endotracheal tube cuff pressure apparatus.

FIG. 7 shows one embodiment of the pressure sensor circuitry of the present invention.

FIG. 8 shows one embodiment of the differential amplifier circuitry of the present invention.

FIG. 9 shows one embodiment of the comparator circuit of the present invention.

FIG. 10 shows an illustration of the process flow used to program the instrument.

### DETAILED DESCRIPTION OF THE INVENTION

The invention relates to methods and devices related to improvements in the use of a medical breathing tube. In some embodiments, the invention reduces injuries and diseases associated with the use of said breathing tube. In preferred embodiments, the present invention provides methods and devices to ensure proper pressure of an endotracheal tube cuff.

In preferred embodiments, the invention relates to the use of an endotracheal tube. The endotracheal tube serves as an open passage through the upper airway. The purpose of endotracheal intubation is to permit air to pass freely to and from the lungs in order to ventilate the lungs. Endotracheal tubes can be connected to ventilator machines to provide artificial respiration. This helps in maintaining the patient's airway, especially during surgery. It is often used when patients are critically ill and cannot maintain adequate respiratory function to meet their needs. The endotracheal tube facilitates the use of a mechanical ventilator in these critical situations. If the tube is inadvertently placed in the esophagus (right behind the trachea), adequate respirations will not occur. Brain damage, cardiac arrest, and death can occur. Aspiration of stomach contents can result in pneumonia and acute respiratory distress syndrome. If the tube is placed too deep, it could result in only one lung being ventilated and can result in a pneumothorax as well as inadequate ventilation. During endotracheal tube placement, damage can also occur to the teeth, the soft tissues in the back of the throat, as well as the vocal cords.

Typically, an endotracheal tube terminates at one end in a coupling for coupling the tube to a supply tube that supplies the ventilating medium source. An inflatable cuff is provided at the other end of the endotracheal tube and extends around the tube so that on inflating of the cuff the endotracheal tube is secured in the trachea of the subject and leak passed of the ventilating medium into the mouth of the subject is avoided during the inspiratory phase of each breathing cycle. As disclosed in U.S. Pat. No. 6,647,984 to O'Dea, hereby incorporated by reference, cuffs are typically inflated by manual manipulation of a syringe to a pressure adequate for retaining the endotracheal tube in the trachea and also for preventing any leaks of the ventilating medium. As provided for in Young et al., Intensive Care Medicine, 337-347 (2007), incorporated herein by reference, the large diameter/high volume low-pressure cuff has been a standard cuff used by practitioners for several decades. There is no tension within the wall of an inflated high volume low pressure cuff, thus all the intra-cuff pressure is transmitted to the tracheal wall, enabling easy monitoring of the tracheal wall pressure by direct measurement. Unfortunately there is an inherent design fault with these high volume, low-pressure cuffs in that they allow pulmonary aspiration to occur even when correctly inflated.

Cuff pressure is a recognized factor in the pathogenesis of tracheal injury; even the high volume/low pressure cuff may cause mucosal damage over a short period. Areas of ciliary denudation and mucosa injury are seen as early as two hours after intubation. Measurement of intracuff pressure represents a simple and reproducible method of assessing the pressure exerted on the tracheal mucosa. The pressure within the tracheal cuff is assumed to be equal to the pressure exerted on tracheal lining because the high volume cuff does not show changes in pressure until it impinges on the tracheal wall as provided for in Vyas et al. (2002) Anesthesiology 57, 266-283, incorporated herein by reference. It has been suggested that the minimum occluding pressure required to achieve and adequate seal and reduce the risk of aspiration is 25 cm H₂O as disclosed in Vyas et al. (2002) Anesthesiology 57, 266-283, and Bernhard et al. (1979) Anesthesiology 50, 366-369, both of which are hereby incorporated by reference. Pressures greater than 25 cm, H₂O up to two hours will denude mucosa down to the basement membrane as provided for in Vyas et al. (2002) Anesthesiology 57, 266-283, and Nordin et al. (1977) Otolaryngologica 345, S7-S56, both of which are hereby incorporated by reference. In this study, this limit was exceeded in 62% of patients. This may be due to inadvertent overinflation or an attempt to achieve an adequate seal in cases in which the initial tube is too small. The size of the tracheal tube is known to affect the intracuff pressure. Tracheal tubes that are much smaller than the trachea will require greater inflation to prevent an air leak and so will exert a higher pressure on the tracheal mucosa. The benefits of high volume-low pressure cuffs are lost by inflating the cuff above the minimum occlusion volume. It was further noted patients on intensive care are exposed to high cuff inflation pressures and hence pressures exerted on the trachea may also be excessive. It also shows that many intensive care units do not measure the cuff pressure regularly. It is recommended, on the basis of this study, that the cuff pressures in the intensive care units should be measured regularly and with any change in patient position or ventilation. Although this particular study recommends 25 cm H₂O as the recommended upper limit for the cuff pressure, various sources of literature indicate that 20-30 cm H₂O is considered to be the generally accepted recommended lower and upper limit for the cuff pressures.

A preferred embodiment of the present invention is a device capable of continuously monitoring the pressure of endotracheal tube cuffs. In one example of the use of the present invention, the device is operably arranged outside of a patient's body and connected to an endotracheal tube cuff that is resides inside said patient's body. The device measures the pressure of the endotracheal tube cuff and then transmits this pressure data to a computer monitored by a caregiver. In a preferred embodiment, the device comprises a display that displays the current pressure value as well as a graph of current and past pressures. The device, in one embodiment, comprises a visual alarm when pressure is either above or below the pressure range of 20-30 or 20-25 cm H₂O. The system further allows for manual inflation and deflation of the cuff with a syringe. This replicates the current method used by medical personnel so that doctors do not have to drastically change their methods to use this device.

In one embodiment, the device utilizes wireless technology to transmit the pressure data from the device to the computer at, for example, a nurses' station, allowing for the device to be used with each intubated patient in an intensive care unit (ICU) without having wires running from their rooms to the nurses' station. In another embodiment, the device functions while operably linked to a USB cable in lieu of wireless operation.

In one embodiment, the present invention contemplates a stand alone device that can be attached to a endotracheal tube cuff via a port, e.g. a device comprising a housing comprising a port (e.g. in a side wall), said housing containing a pressure sensor in fluid communication with said port (e.g. via tubing from said port to said pressure sensor), said pressure sensor capable of generating voltage and in electronic communication with a comparator, said comparator in electronic communication with an alarm, said comparator positioned within said housing, said alarm visible from a surface (e.g. a top surface) of said housing.

### DETAILED DESCRIPTION OF PREFERRED EMBODIMENTS

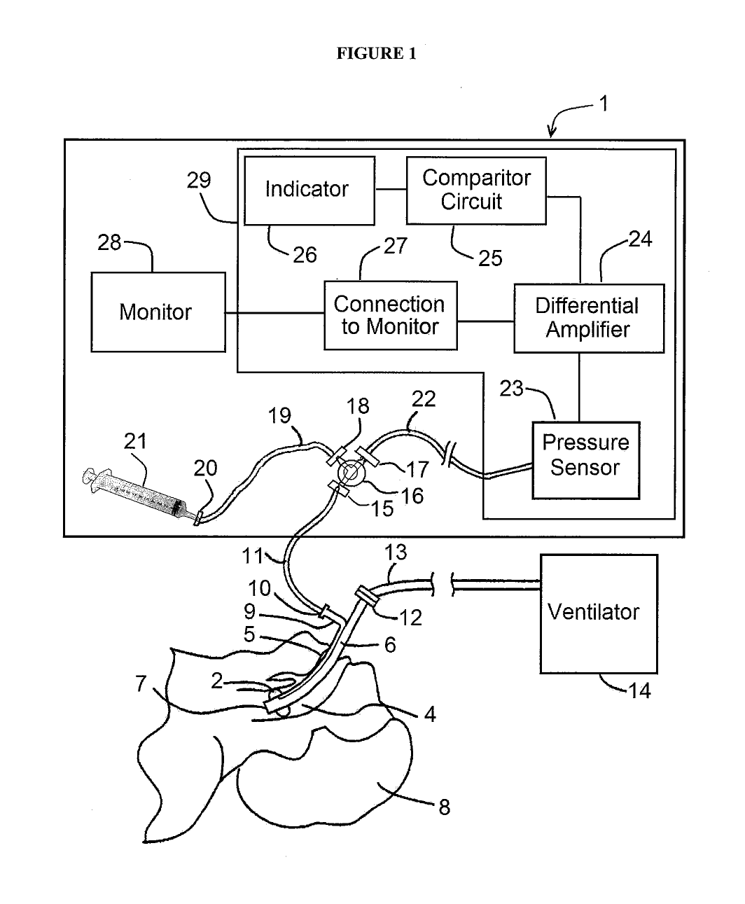

FIG. 1 shows one embodiment of the present invention. The device (indicated generally by the reference numeral 1) for controlling cuff pressure an inflatable cuff 2 of an endotracheal tube 6 comprises a number of elements, which when linked to a ventilation circuit creates a cuff pressure control “coupled system.” The endotracheal tube 6 is inserted through the mouth 5 into the trachea 4 of a subject 8, and a coupling 12 on one end of the endotracheal tube 6 is provided for fluidic communication to a supply tube 13 for in turn connecting in fluidic communication to a ventilator 14. Ventilating medium is supplied to the subject from the ventilator 14. The coupling 12 element may be a “Y tube” or “Y-piece” with arms for the inspiratory line and an expiratory line and described in U.S. Pat. No. 7,334,580, hereby incorporated by reference.

The cuff 2 extends around an end 7 of the endotracheal tube 6. The cuff 2 contacts the walls of the trachea 4, and is inflated for sealing the endotracheal tube 6 in the trachea 4 for preventing leak past of ventilating medium into the mouth of the subject during the inspiratory phase of each breathing cycle. A communicating tube 9 extends along all or part of the endotracheal tube 6, and is (optionally) integrally formed therewith for communicating with the cuff 2 for inflating thereof. The communicating tube 9 terminates in a an inflation valve coupling 10 for connecting the communicating tube 9 and in turn the cuff 2 to the apparatus 1 as will be described. The communicating tube 9 terminates in a first inflation valve coupling 10 which in turn connects via connecting a first delivery tube 11 and terminates in a coupling 15 through a port 30 in the housing 29 which in turn connects in fluidic communication to a three port stopcock/three-way manifold 16. The three port stopcock 16 connects in fluidic communication through coupling 18 in turn through a second delivery tube 19 and terminates in a second inflation valve coupling 20. The syringe 21 connects in fluidic communication via said second inflation valve coupling 20 to the delivery tube 19. The three port stopcock 16 connects in fluidic communication through coupling 17 in turn through a third delivery tube 22 and terminates in a Pressure Sensor 23 (e.g. through a port 31 in the housing 29). In one embodiment, the Pressure Sensor 23 connects electronically to the Differential Amplifier 24. The Differential Amplifier 24 connects electronically to the Comparitor Circuit 25 and in turn to the Indicator 26. The Differential Amplifier 24 communicates electronically through the Connection to Monitor 27 to the Monitor 28. In one embodiment the Pressure Sensor 23, Differential Amplifier 24, Comparator Circuit 25, Indicator 26, and the Connection to Monitor 27 are contained in a Housing 29. The Pressure Sensor 23 sends the information for amplification and that signal is sent to a Comparator Circuit 25 which causes the Indicator 26 to signal (light emitting diodes (LEDs) to light or audio alarm to sound) when the pressure exceeds the desired high or low limits.

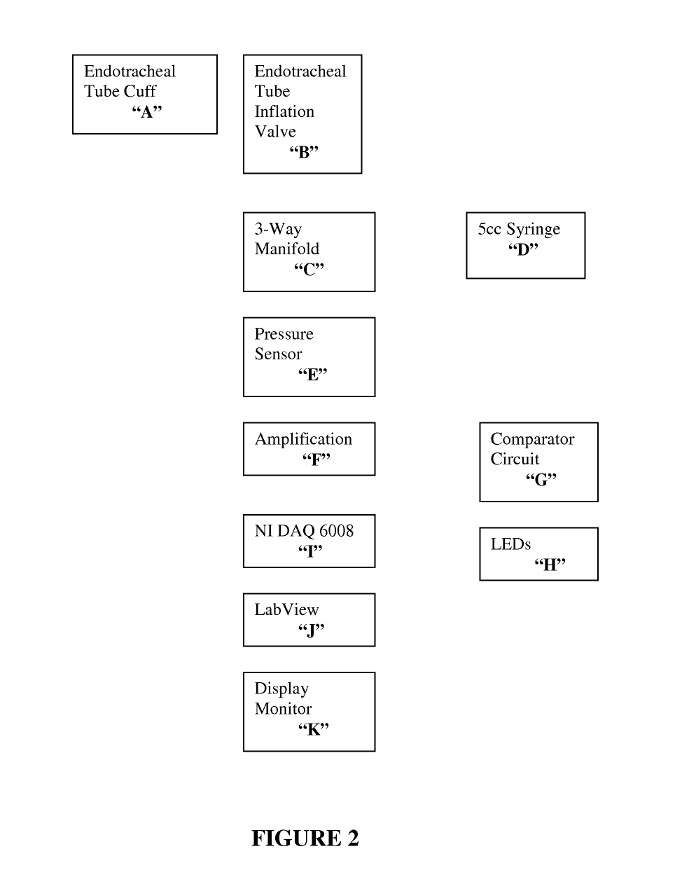

FIG. 2 shows one embodiment of the present invention wherein the indicator 26 displays a solid red light (the light on the right) when the endotracheal cuff pressure is not in the desired range (e.g. less than 20 cm H₂O), while the other light (the light on the left) is not illuminated. FIG. 2 is a photograph of one embodiment of the device of the present invention labeled according to the invention indicated generally by the reference numeral 1 for controlling cuff pressure in an inflatable cuff 2 of an endotracheal tube 6. The endotracheal tube is inserted through the mouth into the trachea of a subject (not shown), and a coupling 12 on one end of the endotracheal tube is provided for connecting the endotracheal tube 6 in fluidic communication to a supply tube (not shown) for in turn connecting in fluidic communication to a ventilator (not shown).

The cuff 2 extends around an end of the endotracheal tube 6 which is inserted in the trachea 4, and is inflated for sealing the endotracheal tube 6 in the trachea for preventing leaks past of ventilating medium into the mouth of the subject during the inspiratory phase of each breathing cycle. A communicating tube 9 extends along all or part of the endotracheal tube 6, and is (optionally) integrally formed therewith for communicating with the cuff 2 for inflating thereof. The communicating tube 9 terminates in a first inflation valve coupling 10 for connecting the communicating tube 9 and in turn the cuff 2 to the apparatus 1 as will be described below. The communicating tube 9 terminates in said first inflation valve coupling 10 which in turn connects via connecting the delivery tube 11 through a port 30 in the housing 29 and terminates in a coupling which in turn connects in fluidic communication to a three port stopcock/three-way manifold (not shown). The three port stopcock connects in fluidic communication through coupling in turn through a delivery tube 19 through a port 31 in the housing 29 and terminates in a second inflation valve coupling 20. The syringe 21 connects in fluidic communication to said second inflation valve coupling 20 to the delivery tube 19. The three port stopcock connects in fluidic communication through coupling in turn through a delivery tube and terminates in a Pressure Sensor (not shown). The Pressure Sensor connects electronically to the Differential Amplifier (not shown). The Differential Amplifier connects electronically to the Comparitor Circuit (not shown) and in turn to the Indicator 26. The Differential Amplifier communicates electronically through the Connection to Monitor to the Monitor (not shown). In one embodiment the Pressure Sensor, Differential Amplifier, Comparator Circuit, Indicator, and the Connection to Monitor are contained in a Housing 29. The a Pressure Sensor sends the information for amplification and that signal is sent to a Comparator Circuit which causes the Indicator to signal (in this embodiment a light emitting diodes (LEDs) to light) when the pressure exceeds the high or low limits.

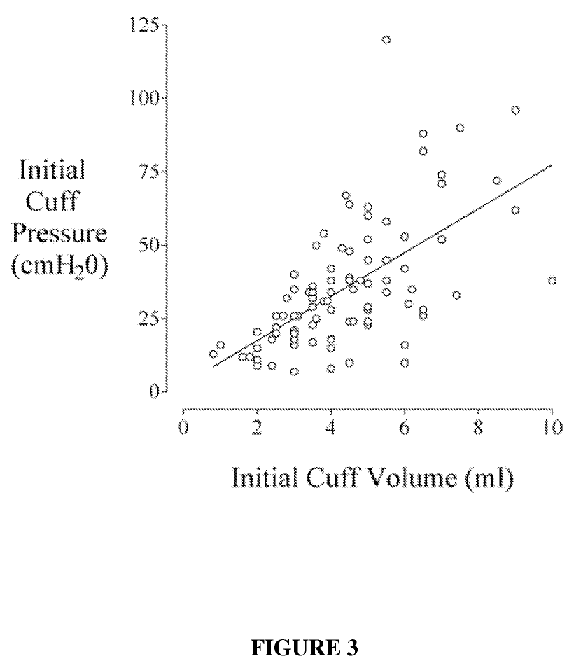

FIG. 3 shows the embodiment of the present invention shown in FIG. 2, except that the indicator displays a solid green light (the light on the left) which shows the endotracheal cuff pressure is between than 20 cm H₂0 and 30 cm H₂O, while the other light (the light on the right) is not illuminated.

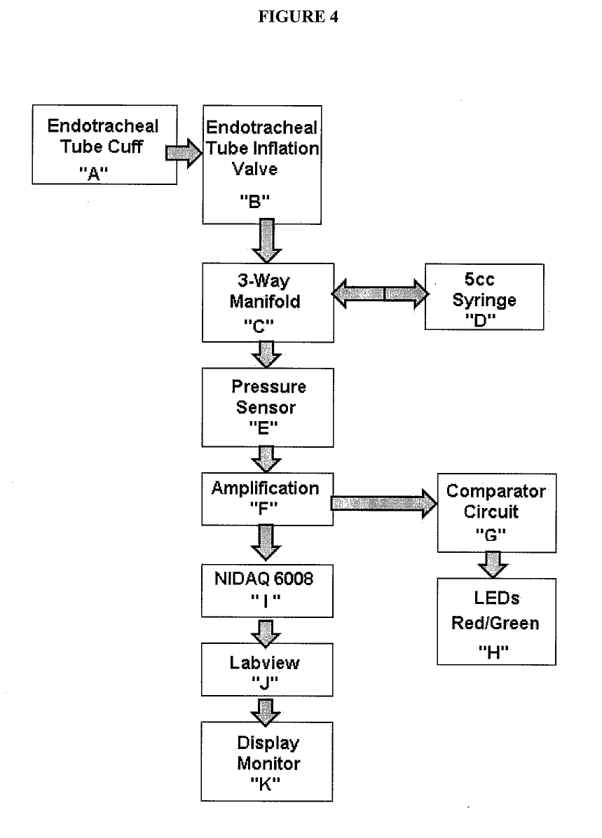

FIG. 4 shows a block diagram detailing the components comprising the device. The endotracheal tube cuff (A) is connected in fluidic communication to the inflation valve of the cuff (B). A three-way manifold (C) is connected in fluidic communication to the cuff inflation valve (B), a 5 cubic centimeter (cc) syringe (D) and to the pressure sensor (E). The pressure sensor sends the information for amplification (F) and that signal is sent to a comparator circuit (G) which causes the light emitting diodes (LEDs) (H) to light when the pressure exceeds the high or low limits. The signal is also sent by the NI DAQ 6008 computer (I) to the monitoring area where LabView program (J) graphs the pressures on the display monitor (K).

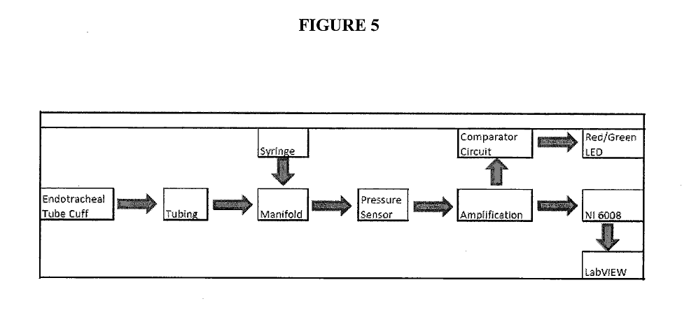

FIG. 5 shows a block diagram detailing the components comprising the device. The endotracheal tube cuff is connected in fluidic communication to the inflation valve of the cuff. A three-way manifold is connected in fluidic communication to the cuff inflation valve, a 5 cubic centimeter (cc) syringe and to the pressure sensor. The pressure sensor sends the information for amplification and that signal is sent to a comparator circuit which causes the light emitting diodes (LEDs) to light when the pressure exceeds the high or low limits. The signal is also sent by the NI DAQ 6008 computer to the monitoring area where LabView program graphs the pressures on the display monitor.

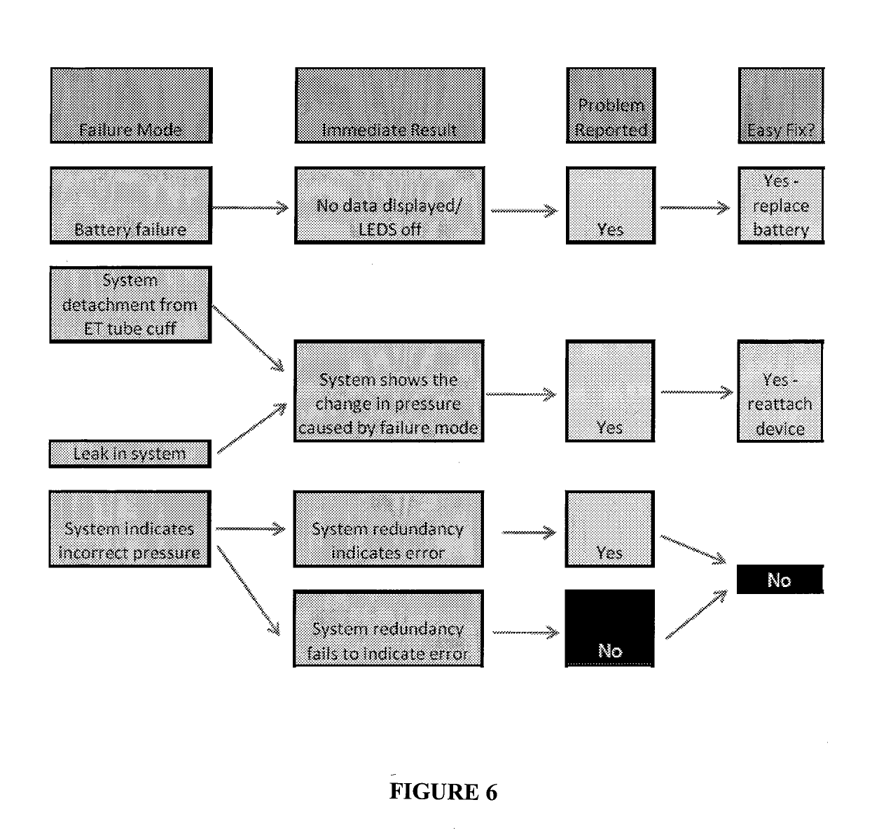

FIG. 6 shows a design flow chart for potential failures and appropriate corrective actions for said failures related to the use of an endotracheal tube cuff pressure apparatus. The problems foreseen for the device include battery failure, accidental disconnection of the device from the endotracheal tube, system leaks, and the system indicating incorrect pressure. In the case of device battery or power source failure, no data will be displayed and the LEDs would be off. The lack of LED lights would alert the medical staff. A battery or power source replacement would fix the issue. In the case disconnection of the device from the endotracheal tube, the risk is minimal as endotracheal tube would remain at the same pressure as was before the device was removed. If the device was removed, the valve connecting the endotracheal tube to the pressure monitoring system would close, sealing off the tube cuff and maintaining its current pressure. When the device becomes detached, it will read and transmit a zero pressure value to the LabVIEW VI (monitoring program), which would cause a visual alarm and prompt investigation by medical personnel. A leak in the system would transmit a zero or low pressure value to the LabVIEW VI (monitoring program), which would cause a visual alarm (red) and prompt investigation by medical personnel. If the system indicates incorrect pressure either system redundancy will indicate the error and report the problem or if it fails to indicate the error there will not be an easy solution to the problem. The potential risks and their solutions have been summarized in FIG. 6. Since the pressure monitoring device is an external device—it is connected to the cuff that is present inside the human body, it could be assumed that the risks posed by the device are not significantly high. Therefore in FDA terminology, the device is not a SR device (Significant Risk device). This would imply that the IDE has to be approved by IRB and a direct additional approval from the FDA may not be necessary.

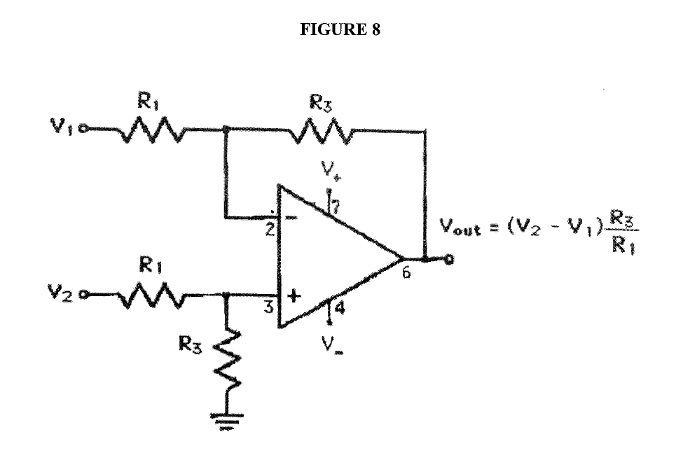

Table 1 shows resistor values for the differential amplifier circuitry seen in FIG. 8. These resistor values amplify the FSO voltage from 3.012 V to approximately 7.03 V. The major benefit of this voltage is relative to the ground, making analysis of this voltage with the comparator circuit possible. This output goes directly to the Vᵢₙ, of the comparator circuit as well as AIO⁺ channel of the NI USB 6008 DAQ.

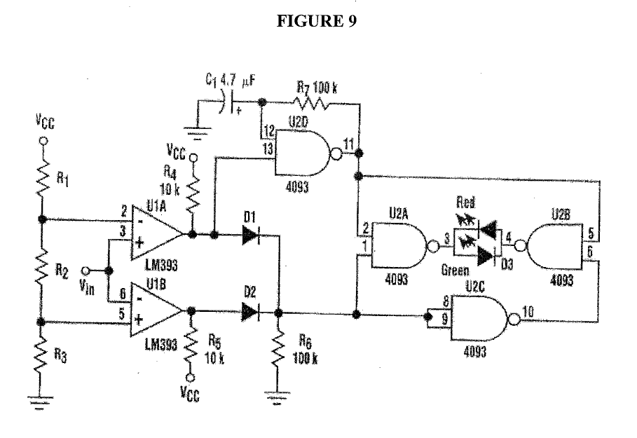

Table 2 shows resistor and capacitor values for the comparator circuit seen in FIG. 9. These values were chosen to give the appropriate voltage window corresponding to the ideal pressure range of 20-30 cm H₂O. The lower bound of this window is 2 V, and the upper bound is 3V. The circuit has a supply voltage (V_{cc}) of 9V, and its input voltage (V₁ₙ) is the amplified voltage from the pressure sensor which ranges from 0 V (at 0 cm H₂O) to 7 V (at 70.31 cm H₂O).

In one embodiment, the Pressure Sensor is a Measurement Specialties Model 1210 (product: 1210A-001G-3S) low pressure sensor used to measure the pressure and convert the signal to voltage. The Model 1210 pressure sensor has a pressure range from 0-1 psi, which is equivalent to 0-70.31 cm H₂O. This range covers the acceptable pressure range of 20-30 cm H₂O and allows for pressure readings from 0 cm H₂O (gauge) to over-inflations by up to 40.31 cm H₂O. Endotracheal tube cuff pressures are not expected to ever exceed 70 cm H₂O. The pressure sensor is made of piezoresistive silicon and is ideal for biomedical applications due to its low pressure range. The pressure from the Endotracheal tube cuff in connected to the tube of the pressure sensor. This tube has an outer radius of 0.12 inches (3.0 mm). It has a pressure non-linearity of only +/−0.2% span, which correlates to about 0.14 cm H₂O, indicating a very accurate pressure reading. The sensor also has internal temperature compensation within a range of 0-50° C. (32-122° F.). Temperature conditions will be well within this range.

In one embodiment a 3 port stopcock/three-way manifold is used to connect in fluidic communication to the inflating/deflating syringe, the pressure sensor, and the tubing/delivery tube leading to the endotracheal tube cuff. This component allows for a continuous pathway of air between all three parts. In one embodiment the two ports have fixed male rotating adapters and connect to the syringe and the pressure sensor tubing. In one embodiment the other port has a fixed female rotating adapter and is connected to tubing coming from the endotracheal tube cuff. In one embodiment the pressure sensor tube and the stopcock/manifold are connected using standard medical grade tubing. In one embodiment the tubing has an inner diameter of 3/32 inches (0.09375 inches) allowing it to be stretched around the pressure sensor tube. In one embodiment the tubing has a fixed male rotating adapter on one end and a fixed female rotating adapter on the other end. In one embodiment the invention is enclosed in a solid housing. In one embodiment the invention is housed in an acrylonitrile butadiene styrene (ABS) plastic enclosure.

In one embodiment, the pressure sensor area of the device employs two operational amplifiers, two 100 kΩ resistors, and a 4.2 kΩ resistor. In one embodiment, the differential amplifier of the device employs one operational amplifier, two 3 kΩ resistors, and two 7 kΩ resistors. In one embodiment, the comparator portion of the circuit used a dual comparator, a quad 2-input NAND Schmitt trigger, a 4.7 μF electrolytic capacitor, a 6 kΩ resistor, 1 kΩ resistor, 2 kΩ resistor, two 101kΩ resistors, and two 100 kΩ resistors.

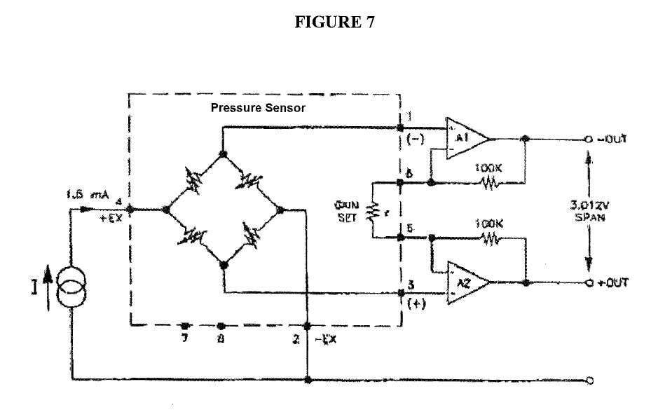

In one embodiment, pressure sensor circuitry of the present invention is shown in FIG. 7. The pressure sensor utilizes two operational amplifiers to amplify its full scale output (FSO) to 3.012V, which corresponds to the pressure at 70.31 cm H₂O. This output from the circuit is a differential voltage, which means that the output nodes are not necessarily 0V and 3.012 V at full scale output. The operational amplifiers are powered with 9V and −9V V_{cc}. Because the pressure sensor circuit outputs a differential voltage, a differential gain amplifier is used.

In one embodiment, the differential amplifier circuitry of the present invention is shown in FIG. 8. The differential amplifier serves primarily to eliminate the non-zero node output by the pressure sensor. The resistor values can be seen in Table 1.

In one embodiment, comparator circuit of the present invention is shown in FIG. 9. The comparator circuit is used separately from the LabView VI to analyze voltages to determine whether or not the endotracheal tube cuff is properly inflated. In one embodiment the comparator circuit uses an LM393 dual comparator and the logic circuit element 4093 NAND Schmitt trigger to light one of the two LEDs depending on the amplified voltage output of the pressure sensor as shown in FIG. 9. The resistor and capacitor values are seen in Table 2. These values were chosen to give the appropriate voltage window corresponding to the ideal pressure range of 20-30 cm H₂O. This lower bound of this window is 2 V, and the upper bound is 3 V. The circuit has a supply voltage (V_{cc}) of 9V and its input voltage (Vᵢₙ) is the amplified voltage from the pressure sensor which ranges from 0 V (at 0 cm H₂O) to 7 V (at 70.31 cm H₂O).

In one embodiment, the comparator has two different output nodes, seen in FIG. 9 as the outputs from U1A and U1B. The output voltages depend on the value of Vᵢₙ. The Schmitt triggers (U2A, U2B, etc.) seen in FIG. 9 use NAND logic to control their outputs. Inputs can either be high (9 V) or low (0 V). When both inputs are high, the trigger output is low; otherwise, the output is high. When the pressure is below 20 cm H₂O, the output of U1B will be V_{cc} (9 V), and the output of U1A will be ground. The input to U2D is 0 V, so its output will always be V_{cc} (9 V). The two inputs to U2A are both 9 V, which makes its output 0V. Both inputs into U2C are then 9 V, which makes its output 0V. The inputs U2B are 0 V and 9 V, which causes it to output 9 V, thus illuminating the red LED. When the press is in the idea range of 20-30 cm H₂O, both U1A and U1B will both output 0 V. Like the under 20 cm H₂O case, U2D will again output 9 V. U2A receives inputs of 0 V and 9 V, causing its output to be 9 V. U2C receives two 0 V inputs, which causes it to output 9 V. U2B receives two 9 V inputs, resulting in a 0 V, which turns on the green LED. When the pressure is in the 20-30 cm H₂O range, the green LED is illuminated indicating proper inflation. When the pressure is above 30 cm of H₂O, the red light blinks due to the constant charging and discharging of the capacitor.

In one embodiment, the data transmission is only the AIO channel of the NI USB-6008 is used. AIO⁺ is connected to the voltage output of the differential amplifier, and AIO⁻ is connected to ground. This component transmits the amplified pressure sensor output voltage to the LabVIEW virtual instrument ad the computer. It performs analog to digital conversion and all other steps necessary for data transmission.

In one embodiment, a National Instruments (NI) USB-6008 multifunction data acquisitions (DAQ) module was selected to transmit the voltage data from the device circuit to a computer with LabVIEW virtual instrument. The USB-6008 has 12-bit input and output resolution and a maximum sampling rate of 10 kS/s. In some embodiments the acquisitions device interfaces with a computer system.

In one embodiment, the LabVIEW virtual instrument takes the voltage data from the device and displays it on a graph of pressure versus time. The pressure voltages range from 0-3.012 V, with 0 V indicating 0 cm H2O and 3.012 V indicating 70.31 cm H2), and the voltage graph is scaled so that these values match. The pressure sensor exhibits a linear relationship between pressure and voltage, so this scaling of the graph converts voltage readings to pressure readings.

In one embodiment, the LabVIEW VI is programmed to obtain and display one pressure value per second. These values are displayed on the pressure versus time graph, and the used has control over how many pressure values are displayed. In one embodiment 120 values or two minutes of pressure history are values to be displayed.

In one embodiment, the virtual instrument also utilizes a visual alarm to alert the user if the endotracheal tube cuff pressures are not with proper pressure range. In one embodiment, the virtual instrument also utilizes a visual alarm to alert the user if the endotracheal tube cuff pressures are not with proper pressure range of 20-30 cm H₂O. In one embodiment, if the pressure falls between 20 and 30 cm H₂O, a green light is displayed indicating proper pressure. In one embodiment, if the pressure rises or falls outside of this range, a red light indicates that the pressure needs to be altered. In one embodiment, the alarm is controlled by the average of the last ten pressure values.

In one embodiment, the virtual instrument also utilizes an audio alarm to alert the user if the endotracheal tube cuff pressures are not with proper pressure range. In one embodiment, the virtual instrument also utilizes an audio alarm to alert the user if the endotracheal tube cuff pressures are not with proper pressure range of 20-30 cm H₂O. In one embodiment, if the pressure falls between 20 and 30 cm H₂O, the alarm is silent indicating proper pressure. In one embodiment, if the pressure rises or falls outside of this range, an audio alarm indicates that the pressure needs to be altered. In one embodiment, the alarm is controlled by the average of the last ten pressure values.

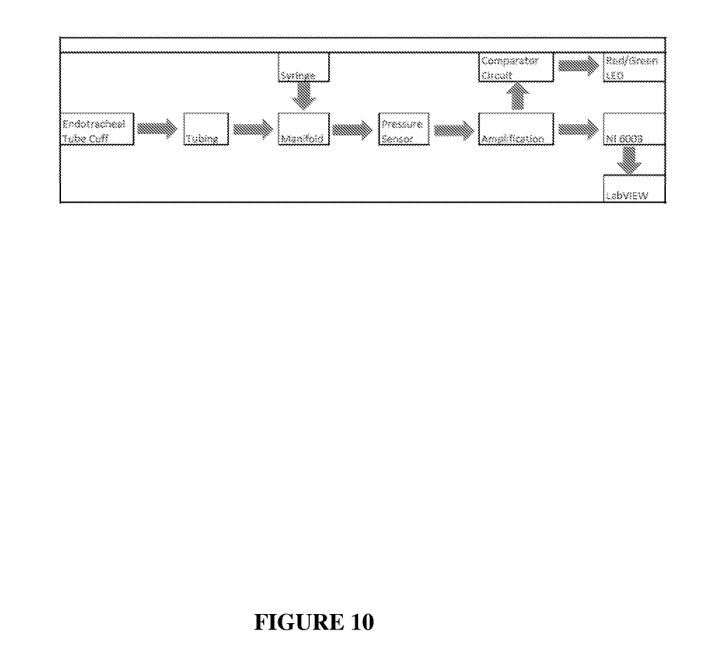

In one embodiment, the LabVIEW virtual instrument is started with a simple on/off switch on the front panel. In one embodiment, the virtual instrument flow chart is represented by in FIG. 10.

TABLE 1
Resistor Values for Differential Amplifier

| | Resistance |
|---|---|
| Resistor | (kΩ) |
| R1 | 3k |
| R3 | 7k |

TABLE 2
Resistor/Capacitor Values for Comparator Circuit

| | Resistance (Ω) or |
|---|---|
| Element | Capacitance (F) |
| R₁ | 6k |
| R₂ | 1k |
| R₃ | 2k |
| R₄ | 10k |
| R₅ | 10k |
| R₆ | 100k |
| R₇ | 100k |
| C₁ | 4.7μ |

What is claimed is:

1. A system comprising: an endotracheal tube cuff in fluid communication with a pressure sensor and a cuff inflating/deflating means, said pressure sensor capable of generating voltage and in electronic communication with a comparator, said comparator in electronic communication with an alarm.

2. The system of claim 1, wherein an amplifier is positioned between said pressure sensor and said comparator, in order that said voltage of said pressure sensor is amplified, and in order that said amplified voltage is transmitted to said comparator as input.

3. The system of claim 1, wherein said comparator is dual comparator.

4. The system of claim 1, wherein said comparator comprises Schmitt triggers.

5. The system of claim 1, wherein said endotracheal tube cuff is attached to a endotracheal tube positioned in a patient.

6. The system of claim 5, wherein said alarm is a visual alarm.

7. The system of claim 6, wherein said alarm is remote from said tube cuff.

8. The system of claim 7, wherein said alarm is visible from a nurse's station.

9. A method comprising: a) continuously monitoring cuff pressure of endotracheal tube cuff with a pressure sensor, said tube cuff attached to a endotracheal tube, said tube positioned in a patient, said pressure sensor capable of generating voltage and in electronic communication with a comparator, said comparator in electronic communication with an alarm; and b) alerting medical staff when the pressure is not optimum with said alarm.

10. The method of claim 9, wherein said alarm is an audible alarm.

11. The method of claim 9, wherein said alarm is a visual alarm.

12. The method of claim 11, wherein said alarm is remote from said tube cuff.

13. The method of claim 12, wherein said alarm is visible from a nurse's station and said medical staff are alerted at said nurse's station.

14. The method of claim 9, wherein said pressure sensor is also in fluid communication with a cuff inflating/deflating means.

15. The method of claim 11, wherein said medical staff adjust said pressure with said inflating/deflating means after being alerted in step b).

16. The method of claim 15, wherein said adjusting comprises pushing or pulling on a syringe, said syringe in fluid communication with said tube cuff.

17. The method of claim 9, wherein said monitoring of step a) comprises generating a voltage with said pressure sensor, and transmitting said voltage to said comparator.

18. The method of claim 10, wherein said medical staff adjust said pressure with said inflating/deflating means after being alerted in step b).

19. The method of claim 18, wherein said adjusting comprises pushing or pulling on a syringe, said syringe in fluid communication with said tube cuff.

20. A device comprising a housing comprising a port, said housing containing a pressure sensor in fluid communication with said port, said pressure sensor capable of generating voltage and in electronic communication with a comparator, said comparator in electronic communication with an alarm, said comparator positioned within said housing, said alarm visible from a surface of said housing.

21. The device of claim 20, further comprising a endotracheal tube cuff attached to said port and in fluid communication with said pressure sensor.
